(19) United States
(12) Patent Application Publication
(10) Pub. No.: US 2026/0183495 A1
(43) Pub. Date: Jul. 2, 2026

(54) INHALER

(71) Applicant: KT&G CORPORATION, Daejeon (KR)

(72) Inventors: Wonkyeong LEE, Daejeon (KR); Hunil LIM, Daejeon (KR); Jinsoo SEONG, Daejeon (KR)

(73) Assignee: KT&G CORPORATION, Daejeon (KR)

(21) Appl. No.: 19/340,601

(22) Filed: Sep. 25, 2025

(30) Foreign Application Priority Data
Dec. 31, 2024 (KR) 10-2024-0202743

(51) Int. Cl. A61M 15/00

(52) U.S. Cl. CPC A61M 15/0031; A61M 15/0001; A61M 15/0036; A61M 15/0038; A61M 15/0041; A61M 2205/3331

(57) ABSTRACT

An inhaler includes a housing including a mounting space to accommodate a cartridge storing target materials and a rupture portion including an airflow passage and configured to rupture the cartridge, wherein the rupture portion includes a first rupture portion and one or more second rupture portions, the first rupture portion extending in a first direction which is a longitudinal direction of the housing and having pointed one end protruding in the mounting space and the each of one or more second rupture portions extending in a second direction crossing the first direction and being movable in the second direction between a first position where the pointed one end protrudes in the mounting space and a second position spaced apart from the mounting space.

### CROSS-REFERENCE TO RELATED APPLICATION

This application is based on and claims priority under 35 U.S.C. § 119 to Korean Patent Application No. 10-2024-0202743, filed on Dec. 31, 2024, in the Korean Intellectual Property Office, the disclosure of which is incorporated by reference herein in its entirety.

### BACKGROUND

### 1. Field

Various embodiments relate to an inhaler, and more particularly, to an inhaler including a powder cartridge rupture portion through which air may pass.

### 2. Description of the Related Art

Studies have been conducted on inhalers that directly deliver target substances to a user's lungs. In this case, a target substance may refer to theanine, caffeine, taurine, nicotine, etc. and may have a fine granule or dry powder form.

The target substance may be stored in a cartridge. In this case, the cartridge may be mounted on an inhaler and used. When the target substance is completely consumed, the target substance may be refilled or the cartridge may be replaced to continue using the inhaler.

Unlike the method of mounting the cartridge, a fixed storage may be disposed in the inhaler. In this regard, because the storage itself may not be replaced, the inhaler may be used by refilling the target materials in the storage when the target materials are completely consumed.

Fields to which the inhaler is applied include electronic cigarettes that store tobacco substances, drug inhalation devices that aerosolize medicinal substances for the treatment of diseases such as asthma or pulmonary diseases, and the like.

### SUMMARY

In the case of an inhaler in which a cartridge storing a target material is mounted, in order for a user to inhale the target material, air needs to flow into the cartridge and then escape to the outside of the cartridge while carrying the target material.

In this regard, when the cartridge is sealed, because there are no separate air inlet and air outlet in the cartridge, the user needs to make a part through which air passes by rupturing the cartridge such as perforating the cartridge. That is, the user needs to make an air inlet and air outlet in the cartridge. Accordingly, at least two ruptures are required for the cartridge. The two ruptures need to be made in different positions on the cartridge.

On the other hand, the user may rupture the cartridge before mounting the cartridge in the inhaler, but in this case, the target material may leak into the rupture part in a process of mounting the cartridge in the inhaler.

Therefore, in terms of use the inhaler, it may be preferable that the cartridge is ruptured either simultaneously with its mounting in the inhaler or after being completely mounted and fixed in place. Accordingly, a component capable of rupturing the cartridge needs to be disposed inside the inhaler.

In this regard, when the component that ruptures the cartridge not only simply ruptures the cartridge but also is disposed inside the cartridge and has a passage for air to enter and exit, unnecessary leakage of the target material may be prevented as much as possible in a series of processes of inhaling the target material after rupturing the cartridge.

Embodiments provide an inhaler with two or more rupture portions capable of rupturing different parts of a cartridge.

Embodiments also provide an inhaler with a rupture portion in which an airflow passage is formed.

The technical problems of the present disclosure are not limited to the above-described description, and other technical problems may be clearly understood by one of ordinary skill in the art from the embodiments to be described hereinafter.

Additional aspects will be set forth in part in the description which follows and, in part, will be apparent from the description, or may be learned by practice of the presented embodiments of the disclosure.

According to an embodiment, an inhaler includes a housing including a mounting space to accommodate a cartridge storing target materials, and a rupture portion including an airflow passage and configured to rupture the cartridge, wherein the rupture portion includes a first rupture portion extending in a first direction, and having pointed one end protruding to the mounting space, wherein the first direction is a longitudinal direction of the housing, and one or more second rupture portions extending in a second direction crossing the first direction and being movable in the second direction between a first position where the pointed one end protrudes into the mounting space and a second position spaced apart from the mounting space.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view of an inhaler according to an embodiment;

FIGS. 2A to 2C are cross-sectional views of the inhaler according to an embodiment taken along a cross-sectional line A-A′ of FIG. 1, for explaining coupling and arrangement relationships of respective components;

FIG. 3 is a diagram for explaining a second rupture portion applied to an inhaler according to an embodiment;

FIGS. 4A to 4F are diagrams for explaining an operation of a push button applied to an inhaler according to an embodiment;

FIGS. 5A and 5B are diagrams for explaining operations of a locking portion and a lever applied to an inhaler according to an embodiment;

FIGS. 6A to 6D are diagrams for explaining operations of an inhaler according to an embodiment;

FIG. 7 is a perspective view of an inhaler according to another embodiment;

FIGS. 8A and 8B are cross-sectional views of the inhaler according to another embodiment taken along a cross-sectional line B-B′ of FIG. 7, for explaining operations of components disposed inside the inhaler;

FIG. 9 is a diagram for explaining an internal operating state during use of an inhaler according to another embodiment; and

FIG. 10A and FIG. 10B are diagrams for explaining a rupture process of a cartridge mounted in an inhaler according to another embodiment and an internal operation state during use of the inhaler.

### DETAILED DESCRIPTION

Hereinafter, the embodiments disclosed in the present specification will be described in detail with reference to the accompanying drawings. The same or similar elements are denoted by the same reference numerals even though they are depicted in different drawings, and redundant descriptions thereof will be omitted. With regard to the description of the drawings, similar reference numerals may be used to refer to similar or related elements.

In the following description, with respect to constituent elements used in the following description, the suffixes “module” and “unit” are used only in consideration of facilitation of description, and do not have mutually distinguished meanings or functions. As used herein, the suffix “module” or “unit” may include a unit implemented in hardware, software, or firmware, and may be used interchangeably with other terms, for example, “logic,” “logic block,” “part,” or “circuitry.” A “module” or a “unit” may be a single integral component, or a minimum unit or part thereof, adapted to perform one or more functions. For example, the “module” or the “unit” may be implemented in the form of an application-specific integrated circuit (ASIC).

In addition, in the following description of the embodiments disclosed in the present specification, a detailed description of known functions and configurations incorporated herein will be omitted when the same may make the subject matter of the embodiments disclosed in the present specification rather unclear. In addition, the accompanying drawings are provided only for a better understanding of the embodiments disclosed in the present specification and are not intended to limit the technical ideas disclosed in the present specification. Therefore, it should be understood that the accompanying drawings include all modifications, equivalents, and substitutions within the scope and spirit of the present disclosure.

It will be understood that although the terms “first”, “second”, etc., may be used herein to describe various components, these components should not be limited by these terms. These terms are only used to distinguish one component from another component.

It will be understood that when a component is referred to as being “connected to” or “coupled to” another component, it may be directly connected to or coupled to another component, or intervening components may be present. On the other hand, when a component is referred to as being “directly connected to” or “directly coupled to” another component, there are no intervening components present.

As used herein, the singular form is intended to include the plural forms as well, unless the context clearly indicates otherwise.

Embodiments as set forth herein may be implemented as software including one or more instructions that are stored in a storage medium (e.g., a memory 17) that is readable by a machine (e.g., the aerosol-generating device 1). For example, a processor (e.g., the controller 12) of the machine (e.g., the aerosol-generating device 1) may invoke at least one of the one or more instructions stored in the storage medium, and may execute the same. This allows the machine to be operated to perform at least one function according to the at least one instruction invoked. The one or more instructions may include code generated by a compiler or code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

In the present disclosure, the directions of the aerosol-generating device 1 may be defined based on the orthogonal coordinate system. In the orthogonal coordinate system, the x-axis direction may be defined as a leftward-rightward direction of the aerosol-generating device 1. The y-axis direction may be defined as a forward-backward direction of the aerosol-generating device 1. The z-axis direction may be defined as an upward-downward direction of the aerosol-generating device 1.

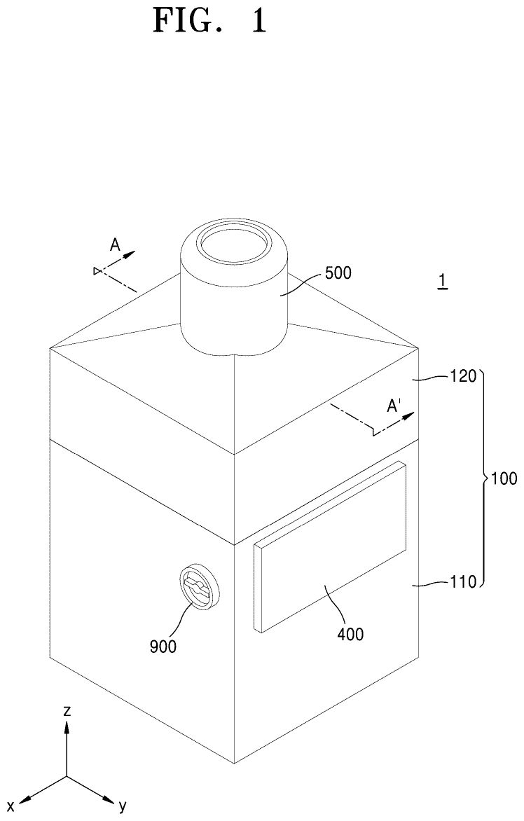

FIG. 1 is a perspective view of an inhaler 1 according to an embodiment.

Referring to FIG. 1, the inhaler 1 according to an embodiment may include a housing 100, a push button 400, a mouthpiece 500, and a lever 900. FIG. 1 illustrates components exposed to the outside of the inhaler 1.

The housing 100 may form the overall exterior of the inhaler 1, and perform a function of accommodating and protecting the components of the inhaler 1. As shown, the housing 100 may have a rectangular parallelepiped shape, but is not limited thereto, and may be manufactured in various shapes such as a cylinder.

The housing 100 may be separated into a lower housing 110 and an upper housing 120. When using the inhaler 1, a user may use the inhaler 1 in the form of the housing 100 in which the lower housing 110 and the upper housing 120 are coupled to each other.

A cartridge storing a target material may be mounted to the inside of the inhaler 1. In this regard, when the target materials stored in the cartridge are completely consumed, the user may replace the cartridge after separating the housing 100 into the lower housing 110 and the upper housing 120.

The push button 400 is a component for moving a rupture portion disposed inside the inhaler 1. The push button 400 may protrude the outside of the housing 100 through a side surface portion of the housing 100. Accordingly, at least a part of the push button 400 is exposed to the outside of the housing 100, so that the user may press the push button 400.

In this regard, a second rupture portion, which will be described below, may be coupled to the push button 400 to move together with the push button 400. As the user presses the push button 400, the second rupture portion connected to the push button 400 may move toward the cartridge and rupture the cartridge.

Because the push button 400 is exposed through the side surface portion of the housing 100, when applying force while gripping the inhaler 1, the user may press the push button 400 while applying the force to the side surface portion of the housing 100.

The mouthpiece 500 is a component disposed on an upper portion of the housing 100 and in contact with a user's mouth. The mouthpiece 500 may be formed to protrude the outside of the housing 100. For example, the mouthpiece 500 may extend in a longitudinal direction (e.g., z-axis direction) of the housing 100 from a region of the upper portion of the housing 100.

The mouthpiece 500 may have a shape that may easily come into contact with the user's mouth. The user may inhale the target material stored in the cartridge after contacting the mouth with the mouthpiece 500.

The lever 900 is a rotatable component according to manipulation of the user. As shown, at least a part of the lever 900 may be exposed to the outside of the housing 100. The user may rotate a locking portion, which will be described below, by manipulating the lever 900 exposed to the outside of the housing 100.

The locking portion engaged with the push button 400 rotates, and thus engagement between the locking portion and the push button 400 may be released. The push button 400 which is no longer supported by the locking portion may move in an outward direction of the housing 100 by pressing of an elastic member and, as shown in FIG. 1, protrude to the outside of the housing 100.

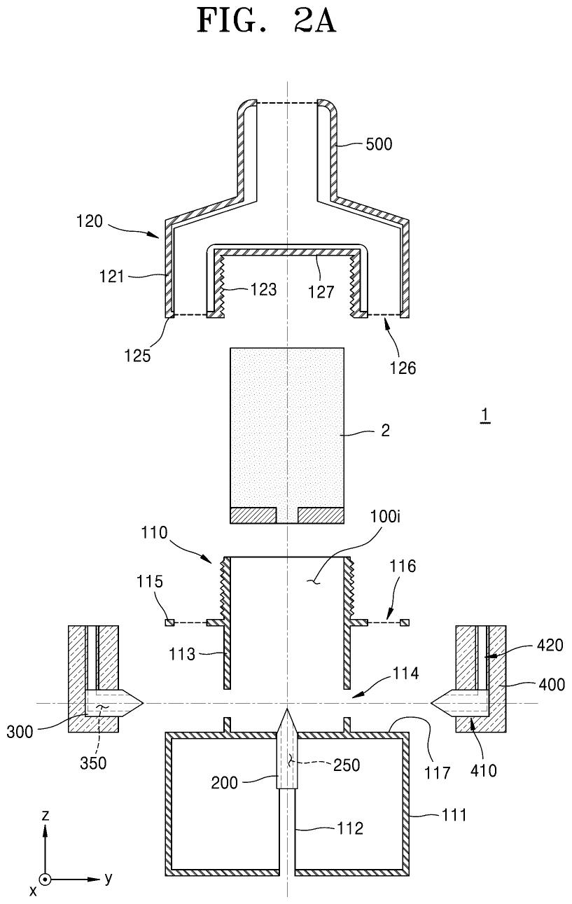

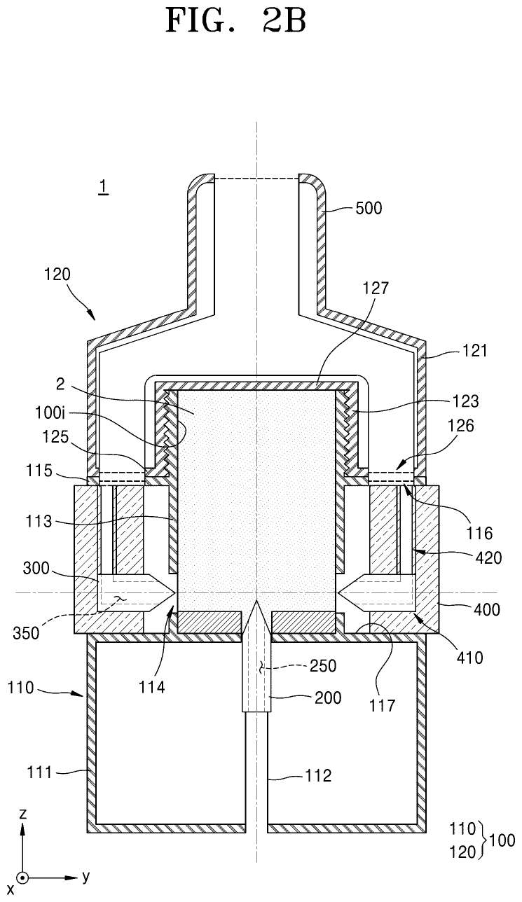

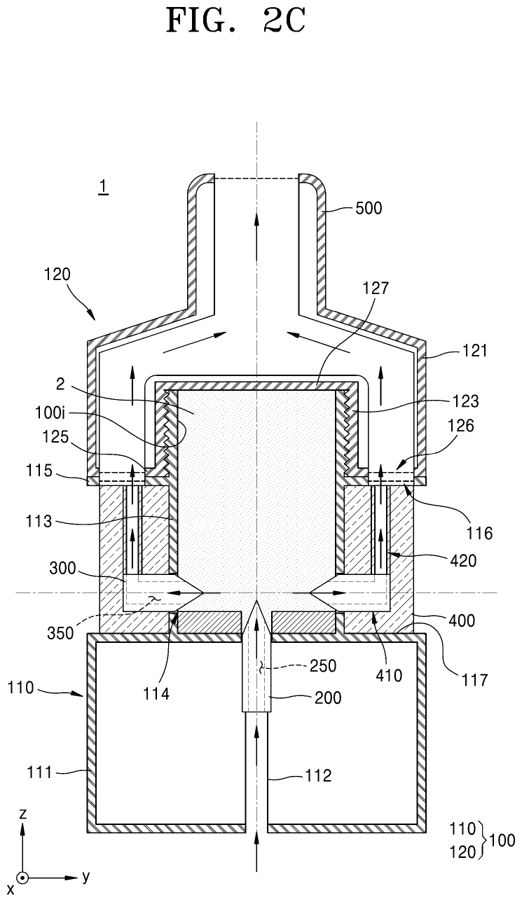

Hereinafter, the components inside the inhaler 1 will be described in detail with reference to FIGS. 2A to 2C.

FIGS. 2A to 2C are cross-sectional views of the inhaler 1 according to an embodiment taken along a cross-sectional line A-A′ of FIG. 1, for explaining coupling and arrangement relationships of the respective components.

FIG. 2A is a cross-sectional view of most of the components of the inhaler 1 which are separated from each other, and FIGS. 2B and 2C are cross-sectional views of the components of the inhaler 1 which are coupled to each other. In this regard, FIG. 2B illustrates a state where a second rupture portion 300 does not rupture a cartridge 2, and FIG. 2C illustrates a state where the second rupture portion 300 ruptures the cartridge 2.

Referring to FIGS. 2A to 2C, the inhaler 1 according to an embodiment may include the housing 100, a first rupture portion 200, the second rupture portion 300, the push button 400, and the mouthpiece 500. Detailed descriptions of the configuration and effects of the inhaler 1 which are redundant with those described above will be omitted.

As described above, the housing 100 may be separated into the lower housing 110 and the upper housing 120. As shown, the cartridge 2 may be disposed between the lower housing 110 and the upper housing 120.

The first rupture portion 200 may be inserted into the lower housing 110 in a first direction which is a longitudinal direction of the housing 100. The second rupture portion 300 and the push button 400 may be inserted into the lower housing 110 in a second direction which is a direction crossing the longitudinal direction of the housing 100. The mouthpiece 500 may be disposed to protrude to an upper side of the upper housing 120.

According to an embodiment, the lower housing 110 may be largely separated into a first part 111, a second part 113, and a third part 115 according to a shape.

In the first part 111 of the lower housing 110, which is a part constituting a lower portion of the housing 100 or the lower housing 110, an air inlet connecting the outside and the inside of the inhaler 1 or the housing 100 may be disposed.

The first part 111 may include an inflow passage 112. The air inlet may be disposed at one end of the inflow passage 112, and the first rupture portion 200 may be disposed at the other end of the inflow passage 112. Accordingly, the outside of the housing 100 and the inside of the cartridge 2 may be fluidly connected to a first airflow passage 250 formed in the first rupture portion 200 by the inflow passage 112. In this regard, ‘fluid connection’ may mean that elements are connected to each other so that a fluid such as air may pass through and flow.

As shown, the inflow passage 112 may extend from the air inlet in the first direction, but the shape of the inflow passage 112 is not limited thereto. The inflow passage 112 may have a complex shape such that a target material stored in the cartridge 2 does not leak to the outside of the housing 100 through the inflow passage 112.

The second part 113 of the lower housing 110 is a part extending from an upper region 117 of the first part 111 to an upper side and forming a mounting space 100i of the cartridge 2. Specifically, the second part 113 may have a cylinder shape with an empty inside, and the empty inside of the second part 113 may be the mounting space 100i of the cartridge 2.

Therefore, the mounting space 100i may be surrounded by the second part 113, a lower portion of the mounting space 100i may be blocked by the upper region 117 of the first part 111, and an upper portion of the mounting space 100i may be opened. In this regard, the mounting space 100i may be opened in the first direction.

The second part 113 may include an opening 114 through which the second rupture portion 300 may pass. As the second rupture portion 300 moves in the direction crossing the longitudinal direction of the housing 100 or in a second direction (e.g., y-axis direction), the opening 114 may also be opened in the second direction.

The third part 115 of the lower housing 110 may extend from one region of the second part 113 in the outward direction of the housing 100 and form an accommodation space accommodating the push button 400 together with the upper region 117 of the first part 111 and the second part 113. In this regard, the accommodation space may be opened in the outward direction of the housing 100 or in the second direction.

The third part 115 may guide the movement of the push button 400 together with the upper region 117 of the first part 111. The upper region 117 of the first part 111 may serve as the ‘bottom surface 117 of the accommodation space’, and the third part 115 may serve as the 'ceiling 115 of the accommodation space'.

The third part 115 may include an opening 116 facing the longitudinal direction of the housing 100 or the first direction. When the push button 400 is accommodated in the accommodation space, the opening 116 may be aligned with a second opening part 420 of the push button 400 to be described below.

Meanwhile, the second part 113 may be separated into an upper portion and a lower portion with respect to the third part 115. The upper portion of the second part 113 may be engaged with the upper housing 120. For example, a thread may be disposed along an outer surface of the upper portion of the second part 113 so that the upper portion of the second part 113 and the upper housing 120 may be coupled to each other. An opening 114 that is opened to face the accommodation space accommodating the push button 400 may be disposed in the lower portion of the second part 113.

According to an embodiment, the upper housing 120 may also be largely separated into a first part 121, a second part 123, a third part 125, and a fourth part 127 according to a shape.

The first part 121 of the upper housing 120 is a part forming the exterior of the upper portion of the housing 100, and the exterior of the housing 100 may include the first part 111 of the lower housing 110 and the first part 121 of the upper housing 120.

The second part 123 of the upper housing 120, which is a component disposed in an inner side of the first part 121, may have a hollow cylindrical shape. A thread may be disposed on an inner surface of the second part 123. Therefore, the second part 113 of the lower housing 110 and the second part 123 of the upper housing 120 may be screw-coupled through the thread. A space between the inner surface of the first part 121 and the outer surface of the second part 123 may be a passage through which air moves.

The third part 125 of the upper housing 120 is a component connecting the first part 121 to the second part 123 in the second direction, and the third part 115 of the lower housing 110 and the third part 125 of the upper housing 120 may be in contact with each other.

The third part 125 may include an opening 126 facing the longitudinal direction of the housing 100 or the first direction. The opening 126 of the third part 125 of the upper housing 120 may be aligned with the opening 116 of the third part 115 of the lower housing 110.

The fourth part 125 of the upper housing 120 is a component that closes an upper portion of the second part 123. Accordingly, the fourth part 125 may close the upper portion of the mounting space 100i, and support the cartridge 2 accommodated in the mounting space 100i so as not to deviate from the mounting space 100i.

In summary, the lower housing 110 may surround at least a part of the outer surface of the cartridge 2 through the second part 113, the upper housing 120 may surround the upper portion of the cartridge 2 through the fourth part 127, and the upper region 117 of the first part 111 of the lower housing 110 may support the lower portion of the cartridge 2.

As a result, the mounting space 100i may be closed by the housing 100, and the cartridge 2 mounted in the inhaler 1 may be protected by being surrounded in all directions by the housing 100.

The rupture portions 200 and 300, which are components for rupturing the cartridge 2, may rupture different parts of the cartridge 2. In the disclosure, each of the rupture portions 200 and 300 may perforate the cartridge 2 through pointed one end, but a method of the rupture portions 200 and 300 rupturing the cartridge 2 is not limited thereto.

According to an embodiment, the rupture portions 200 and 300 may respectively include the airflow passages 250 and 350. Even when the pointed one end of each of the rupture portions 200 and 300 remains inserted into the cartridge 2, air may enter and exit the cartridge 2 through the airflow passages 250 and 350of the rupture portions 200 and 300. Accordingly, it is not necessary to separate the rupture portions 200 and 300 from the cartridge 2 until the target materials stored in the cartridge 2 are completely consumed.

That is, the airflow passages 250 and 350are respectively disposed in the rupture portions 200 and 300, and thus a phenomenon in which the target material moves along the rupture portions 200 and 300 which are separated from the cartridge 2 and unnecessarily leaks may be completely prevented.

According to an embodiment, the rupture portions 200 and 300 may include the fixed first rupture portion 200 and the movable second rupture portion 300.

The first rupture portion 200, which is a fixed component, may extend in the first direction which is the longitudinal direction of the housing 100. The pointed one end of the first rupture portion 200 may protrude into the mounting space 100i. Accordingly, when the user mounts the cartridge 2 in the mounting space 100i, the first rupture portion 200 may rupture the cartridge 2.

The other end of the first rupture portion 200 may be disposed toward the inflow passage 112. When an airflow passage of the first rupture portion 200 is referred to as the first airflow passage 250, the first airflow passage 250 may extend in a longitudinal direction of the first rupture portion 200.

The first airflow passage 250 extending in the first direction may fluidly connect the inside of the cartridge 2 to the inflow passage 112. Therefore, air flowing into the inflow passage 112 from the outside of the housing 10 may pass through the first airflow passage 250 and flow into the cartridge 2.

The second rupture portion 300, which is a movable component, may extend in the second direction which is the direction crossing the longitudinal direction of the housing 100. The second rupture portion 300 may move in the second direction between a first position at which the pointed one end protrudes into the mounting space 100i and a second position at which the pointed one end is spaced apart from the mounting space 100i.

Referring to FIG. 2B, the second rupture portion 300 is at the second position. Because the pointed one end of the second rupture portion 300 is spaced apart from the mounting space 100i, the second rupture portion 300 may not rupture the cartridge 2.

In this regard, because the cartridge 2 was ruptured only by the first rupture portion 200, the cartridge 2 is not fluidly connected to the mouthpiece 500. That is, even when the user attempts to inhale air through the mouthpiece 500, the target material stored inside the cartridge 2 may not move to the mouthpiece 500.

Referring to FIG. 2C, the second rupture portion 300 is at the first position. When the cartridge 2 is accommodated in the mounting space 100i, when the pointed one end of the second rupture portion 300 protrudes into the mounting space 100i, the second rupture portion 300 may rupture the cartridge 2. That is, when the user moves the second rupture portion 300 to the first position, the second rupture portion 300 may rupture the cartridge 2 accommodated in the mounting space 100i.

Unlike rupturing the cartridge 2 when the first rupture portion 200 mounts the cartridge 2 in the mounting space 100i, the second rupture portion 300 may rupture the cartridge 2 after the cartridge 2 is accommodated in the mounting space 100i. That is, the rupture by the first rupture portion 200 may first occur, and then the rupture by the second rupture portion 300 may occur.

The other end of the second rupture portion 300 may be disposed inside the push button 400. When an airflow passage of the second rupture portion 300 is referred to as the second airflow passage 350, the second airflow passage 350 may extend in a longitudinal direction of the second rupture portion 300. As shown, a part of the second airflow passage 350 may extend in the first direction so that an upper portion of the second rupture portion 300 may be opened, but the embodiment is not limited to the corresponding structure.

The push button 400 may include a first opening part 410 that is opened in the second direction and a second opening part 420 that is opened in the first direction. The first opening part 410 and the second opening part 420 may be physically connected to each other to be fluidly connected.

The second rupture portion 300 may be inserted into the push button 400 through the first opening part 410. Specifically, a part of the second rupture portion 300 including the other end of the second rupture portion 300 may be inserted into the first opening part 410.

As shown, the second rupture portion 300 may be inserted to the end of the first opening part 410, so that the second airflow passage 350 may be directly fluidly connected to the second opening part 420. However, when the other end of the second airflow passage 350 is located in the first opening part 410, the second airflow passage 350 may be fluidly connected to the second opening part 420 through the first opening part 410.

In this regard, a degree to which the second rupture portion 300 is inserted into the push button 400 through the first opening part 410 may determine where the other end of the second rupture portion 300 is located. In addition, the shape of the second airflow passage 350 may determine where the other end of the second airflow passage 350 is located.

The second airflow passage 350 extending in the second direction may fluidly connect the inside of the cartridge 2 to the first opening part 410 or the second opening part 420 of the push button 400. The air flowing into the cartridge 2 may pass through the second airflow passage 350 while carrying the target material stored in the cartridge 2 and move to the second opening part 420.

The second opening 420 of the push button 400 may be aligned with the opening 116 of the lower housing 110 and the opening 126 of the upper housing 120 in the first direction. As shown, the second opening part 420 may be aligned with the two openings 116 and 126 regardless of the positions of the second rupture portion 300 and the push button 400. That is, even though the second rupture portion 300 and the push button 400 moving between the first position and the second position are at any positions between the two positions, the second opening part 420 may be aligned with the two openings 116 and 126 regardless of the positions. However, the embodiment is not limited to illustration.

The air flowing into the second opening part 420 may pass through the opening 116 of the lower housing 110 and the opening 126 of the upper housing 120 to flow into the upper housing 120. The air moving along the shape of the inside of the upper housing 120 may reach the mouthpiece 500 and be inhaled by the user.

In summary, when the first rupture portion 200 and the second rupture portion 300 rupture the cartridge 2, air may sequentially pass through the inflow passage 112, the first airflow passage 250, the cartridge 2, the second airflow passage 350, the first opening part 410, the second opening part 420, the inner space of the upper housing 120, and the mouthpiece 500 from the outside of the housing 100 and be inhaled by the user.

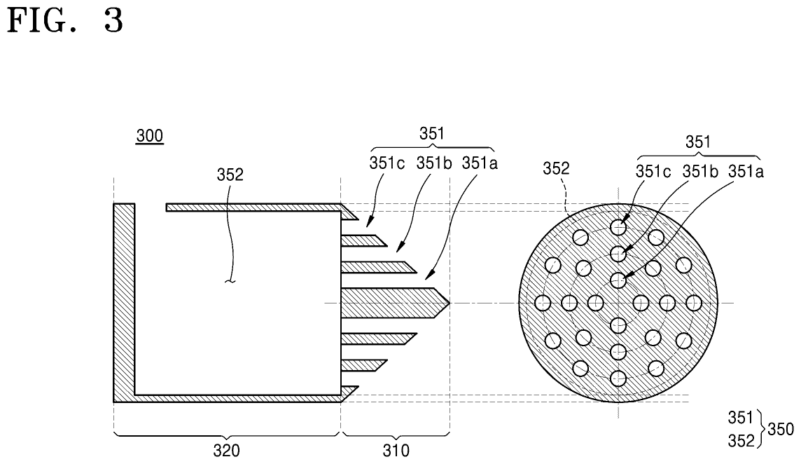

Meanwhile, according to an embodiment, the amount of air passing through the second airflow passage 350 may vary depending on the degree to which the second rupture portion 300 is inserted into the cartridge 2. Hereinafter, the structures of the second rupture portion 300 and the second airflow passage 350 will be described with reference to FIG. 3.

FIG. 3 is a diagram for explaining the second rupture portion 300 applied to an inhaler according to an embodiment. In FIG. 3, the left is a cross-sectional view of the second rupture portion 300, and the right is a side view of the second rupture portion 300.

The second rupture portion 300 may include a rupture part 310 that narrows toward a pointed one end of the second rupture portion 300 and an extension part 320 extending with the same diameter as the other end of the second rupture portion 300. At least a part of the rupture part 310 may be inserted into a cartridge (e.g., the cartridge 2 of FIG. 2A) as the second rupture portion 300 moves in a second direction. That is, when the second rupture portion 300 is at a first position, at least a part of the rupture part 310 may be inserted into the cartridge 2.

The second airflow passage 350 of the second rupture portion 300 may include one or more narrow airflow passages 351 extending from one end of the rupture part 310 in the second direction, and a wide airflow passage 352 extending toward one end of the extension part 320 and combined with the one or more narrow airflow passages 351.

As the rupture part 310 is inserted into the cartridge 2 when the second rupture portion 300 is at the first position, at least one of the narrow airflow passages 351 may start inside the cartridge 2. That is, at least one of ends of the plurality of narrow airflow passages 351 exposed to the outside of the second rupture portion 300 may be located inside the cartridge 2.

Air passing through a first rupture portion (e.g., the first rupture portion 200 of FIG. 2A) and flowing into the cartridge 2 may flow into the second airflow passage 350 of the second rupture portion 300 while carrying a target material stored in the cartridge 2. In this regard, the air may first pass through the one or more narrow airflow passages 351, pass through the wide airflow passage 352 connected to the other ends of the narrow airflow passages 351, and then escape to the outside of the second rupture portion 300.

In this regard, one end of the wide airflow passage 352 may be connected to the other ends of the one or more narrow airflow passages 351, and the other end of the wide airflow passage 352 may be fluidly connected to the outside of the second rupture portion 300.

As shown, the wide airflow passage 352 extends in the second direction, but the other end of the wide airflow passage 352 is opened in a first direction which is a direction crossing a longitudinal direction of the second rupture portion 300. However, the embodiment is not limited to that illustrated. When the wide airflow passage 352 is capable of being fluidly connected to an opening part (e.g., the opening parts 410 and 420 of FIG. 2A) of the push button 400 through the other end, the other end of the wide airflow passage 352 may be opened in the second direction which is the longitudinal direction of the second rupture portion 300.

According to an embodiment, the plurality of narrow airflow passages 351 may be disposed. The plurality of narrow airflow passages 351 may include a first passage 351a starting at a position spaced apart from a pointed one end of the rupture part 310 by a first distance, a second passage 351b starting at a position spaced apart from the pointed one end by a second distance greater than the first distance, and a third passage 351c starting at a position spaced apart from the pointed one end by a third distance greater than the second distance.

Accordingly, as the second rupture portion 300 moves toward the cartridge 2 from the second position, one end of the first passage 351a may be first located inside the cartridge 2, one end of the second passage 351b may be located inside the cartridge 2, and then one end of the third passage 351c may be finally located inside the cartridge 2.

Each of the first passage 351a, the second passage 351b, and the third passage 351c may be disposed in plural. For example, the plurality of first passages 351a may all start at positions spaced apart from the pointed one end of the rupture part 310 by the first distance.

According to an embodiment, the number of second passages 351b may be greater than or equal to the number of first passages 351a, and the number of third passages 351c may be greater than or equal to the number of second passages 351b.

As shown, four first passages 351a may be disposed, eight second passages 351b may be disposed, and twelve third passages 351c may be disposed. However, the number and arrangement of the narrow airflow passages 351 are not limited thereto.

In summary, as a user moves the second rupture portion 300 toward the cartridge 2 through the push button 400, the number of narrow airflow passages 351 starting inside the cartridge 2 increases, and the air flowing into the second airflow passage 350 may gradually increase.

When the amount of air flowing into the second airflow passage 350 is not desired to be further increased, the second rupture portion 300 needs to be fixed to the current position. In addition, when target materials stored in the cartridge 2 are completely consumed, it is necessary to separate the second rupture portion 300 inserted into the cartridge 2 from the cartridge 2 before replacing the cartridge 2. In this regard, the second rupture portion 300 that has been fixed needs to be moved to the second position.

According to an embodiment, the user may control the movement of the second rupture portion 300 coupled to the push button 400 by controlling the movement of the push button 400. Hereinafter, the structures and operations of the push button 400 and its peripheral components will be described.

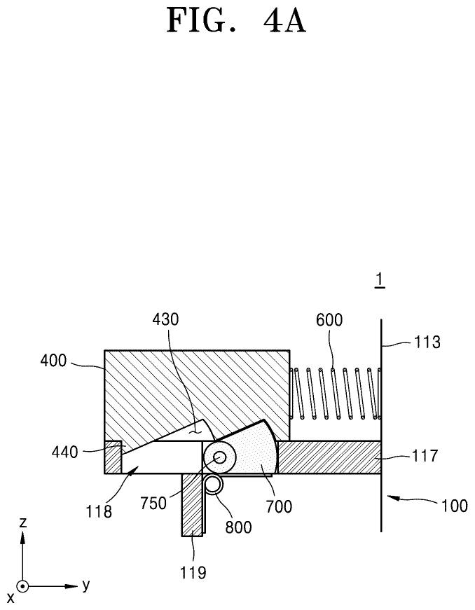

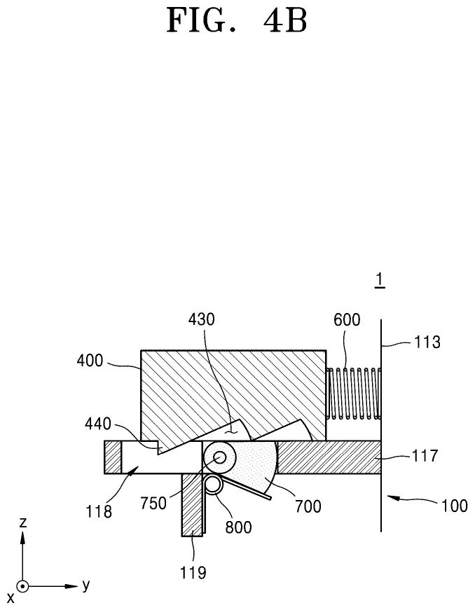

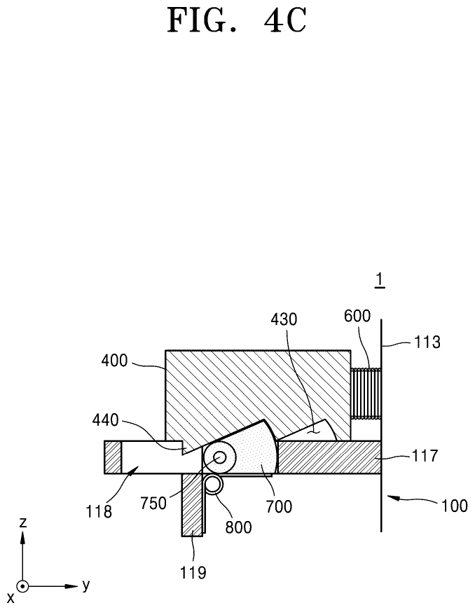

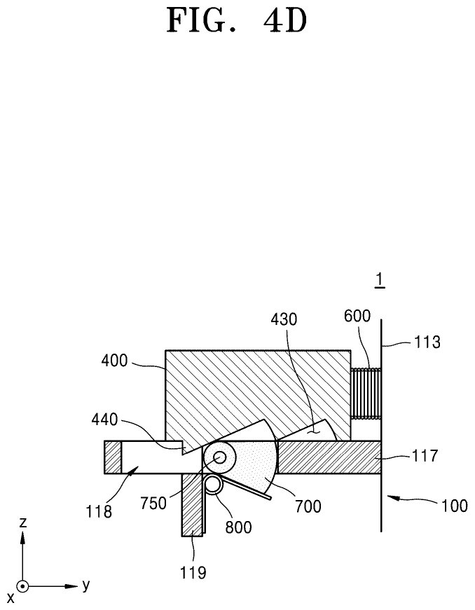

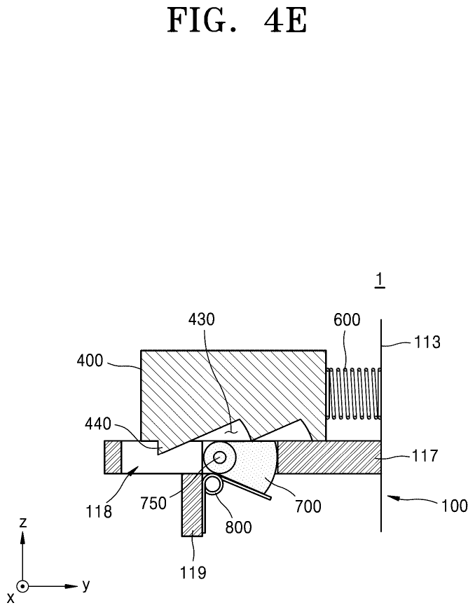

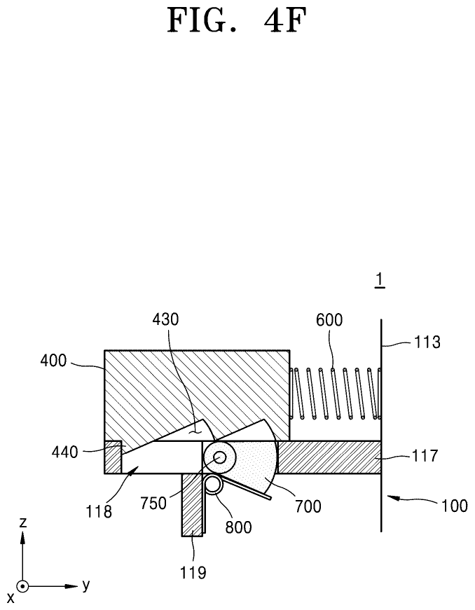

FIGS. 4A to 4F are diagrams for explaining an operation of the push button 400 applied to the inhaler 1 according to an embodiment.

Referring to FIGS. 4A to 4F, the inhaler 1 according to an embodiment may include the housing 100, the push button 400, a first elastic member 600, a locking portion 700, and a second elastic member 800. Detailed descriptions of the configuration and effects of the inhaler 1 which are redundant with those described above will be omitted.

For convenience of description, the internal components of the inhaler 1 are simplified and illustrated. A second rupture portion (e.g., the second rupture portion 300 in FIG. 2A) coupled to the push button 400 is omitted, and only a part of the housing 100 is shown. For example, the second part 113 of a lower housing surrounding a mounting space (e.g., the mounting space 100i of FIG. 2A), the upper region 117 of a first part which is a bottom surface of an accommodation space accommodating the push button 400, and a fourth part 119 extending from the upper region 117 downward. Hereinafter, the upper region 117 of the first part of the lower housing may be expressed as the ‘bottom surface’.

The bottom surface 117 may include a long hole 118 in which a part of the push button 400 and the locking portion 700 are disposed. That is, a part of the push button 400 and the locking portion 700 may pass through the bottom surface 117 in a longitudinal direction of the housing 100 through the long hole 118.

The first elastic member 600 is a component for pushing the push button 400 in an outward direction of the housing 100. That is, the push button 400 may protrude to the outside of the housing 100 by pressing of the first elastic member 600.

One end of the first elastic member 600 may be connected to one region (e.g., the second part 113 of the lower housing) of the housing 100 surrounding the mounting space 100i, and the other end of the first elastic member 600 may be connected to the push button 400.

The locking portion 700 is a component protruding on a movement path of the push button 400 in order to fix the push button 400. That is, at least a part of the locking portion 700 may protrude into the accommodation space of the push button 400.

In response to the locking portion 700, the push button 400 may include one or more grooves 430 engaged with the locking portion 700. The groove 430 may have a shape corresponding to the shape of the locking portion 700. As shown, two grooves 430 are disposed. Accordingly, the push button 400 is fixed to two positions. According to an embodiment, when it is necessary to further subdivide positions to which the push button 400 is fixed, more grooves 430 may be disposed.

The locking portion 700 may move between a ‘closed position’ protruding on the movement path of the push button 400 and fixing the push button 400 and an ‘open position’ spaced apart from the movement path of the push button 400 and allowing the push button 400 to move.

According to an embodiment, the inhaler 1 may further include a rotation shaft 750. The rotation axis 750 is an axis which is the center of rotation of the locking portion 700 and extends in a third direction (e.g., x-axis direction) across the first direction (e.g., z-axis direction) and the second direction (e.g., y-axis direction).

The locking portion 700 may rotate between the closed position and the open position with respect to the rotation shaft 750. That is, a rotation range of the locking portion 700 with respect to the rotation shaft 750 may be between the closed position and the open position.

The second elastic member 800 is a component to press the locking portion 700 to remain protruding on the movement path of the push button 400. That is, the second elastic member 800 may press the locking portion 700 to be at the closed position. Accordingly, as long as a separate external force is applied to the locking portion 700, the locking portion 700 may be basically at the closed position.

One end of the second elastic member 800 may be connected to the fourth part 119 of the lower housing, and the other end of the second elastic member 800 may be disposed in a lower portion of the locking portion 700. Because the locking portion 700 rotates with respect to the rotation shaft 750, the second elastic member 800 may correspond to a torsion spring in response thereto. A first spring arm of the second elastic member 800 which is the torsion spring may be connected to the fixed fourth part 119, and a second spring arm may be connected to the movable locking portion 700, so that the second elastic member 800 may press the locking portion 700 to rotate in a counterclockwise direction.

Referring to FIG. 4A, the groove in the right (hereinafter referred to as the right groove 430) among the two grooves 430 formed in the push button 400 and the locking portion 700 are engaged with each other. The locking portion 700 may be at the closed position by the second elastic member 800.

Referring to FIG. 4B, the user presses the push button 400 to the right and the push button 400 moves. In this case, the locking portion 700 may be pressed by the shape of the push button 400 to rotate in a clockwise direction. Accordingly, the locking portion 700 may be at the open position. The locking portion 700 may remain at the open position until the pressing on the locking portion 700 according to the movement of the push button 400 is released.

Referring to FIG. 4C, the groove in the left (hereinafter, referred to as the left groove 430) among the two grooves 430 formed in the push button 400 and the locking portion 700 are engaged with each other. As the push button 400 moves to the right, when the left groove 430 is aligned at a position corresponding to the locking portion 700 (e.g., when the left groove 430 is located in the upper portion of the locking portion 700), the pressing on the locking portion 700 may be momentarily released, and the locking portion 700 at the open position may be pressed in a counterclockwise direction by the second elastic member 800 to be engaged with the left groove 430. The locking portion 700 may be at the closed position again by the second elastic member 800. In this case, even when the compressed first elastic member 600 presses the push button 400 to the left, because the left groove 430 and the locking portion 700 are engaged with each other, the push button 400 may be fixed without moving.

Referring to FIG. 4d, the locking portion 700 is at the open position by applying an external force to the locking portion 700 engaged with the left groove 430 and rotating the locking portion 700 in a clockwise direction. In this regard, as the compressed first elastic member 600 presses the push button 400 to the left, the push button 400 may move to the left.

Referring to FIG. 4e, the compressed first elastic member 600 presses the push button 400 to the left, and the push button 400 moves. In this regard, because the locking portion 700 is pressed by the shape of the push button 400, the locking portion 700 may remain at the open position even when the external force applied to the locking portion 700 is removed. However, in the disclosure, it is assumed that the external force is still applied to the locking portion 700 until the push button 400 returns to its original position. That is, the locking portion 700 may remain at the open position by the external force.

Referring to FIG. 4F, as the push button 400 moves to the left by the compressed first elastic member 600, the right groove 430 is aligned at a position corresponding to the locking portion 700 (e.g., the right groove 430 is located in the upper portion of the locking portion 700). However, as the external force is still applied to the locking portion 700, the locking portion 700 is still at the open position. In this regard, a structure capable of restricting the movement of the push button 400 toward the left is necessary so that the right groove 430 of the push button 400 may be aligned at the position corresponding to the locking portion 700.

The push button 400 may include a protrusion 440 protruding into the long hole 118 of the bottom surface 117. When the right groove 430 is aligned at the position corresponding to the locking portion 700, the protrusion 440 may reach one end of the long hole 118 and its movement may be restricted. Therefore, when the movement of the push button 400 is restricted by the protrusion 440 and the push button 400 no longer moves, the right groove 430 of the push button 400 may be aligned at the position corresponding to the locking portion 700.

When the external force applied to the locking portion 700 is removed, the locking portion 700 may move back to the closed position by the second elastic member 800. As a result, the right groove 430 of the push button 400 and the locking portion 700 may be engaged with each other. This is the same as that illustrated in FIG. 4A.

In other words, FIGS. 4A to 4F illustrate a process in which the user presses the push button 400 to move the second rupture portion 300 toward the cartridge 2, a process in which the push button 400 and the second rupture portion 300 are fixed to a specific position, and a process in which the push button 400 and the second rupture portion 300 are moved to be away from the cartridge 2 again.

In a process in which the second rupture portion 300 moves toward the cartridge 2, the second rupture portion 300 may rupture the cartridge 2. When the second rupture portion 300 further moves toward the cartridge 2 while the cartridge 2 is ruptured, the cartridge 2 may be ruptured to be larger in correspondence to the shape and size of the rupture part 310 inserted into the cartridge 2. Accordingly, the user may increase the amount of inhaled target materials.

When the push button 400 and the second rupture portion 300 are fixed to the specific position, the amount of air flowing into a second airflow passage (e.g., the second airflow passage 350 of FIG. 3) may no longer increase. That is, when the user inhales air once, the amount of target materials inhaled by the user together with the air may be maintained.

The process of moving the second rupture portion 300 so as to be away from the cartridge 2 may mean a process of separating the second rupture portion 300 inserted into the cartridge 2 from the cartridge 2. This process may proceed before removing the cartridge 2 mounted in the inhaler 1 when the target materials stored in the cartridge 2 are completely consumed and the cartridge 2 needs to be replaced. The second rupture portion 300 needs to be separated from the cartridge 2, in order for the user to separate the cartridge 2 from the inhaler 1.

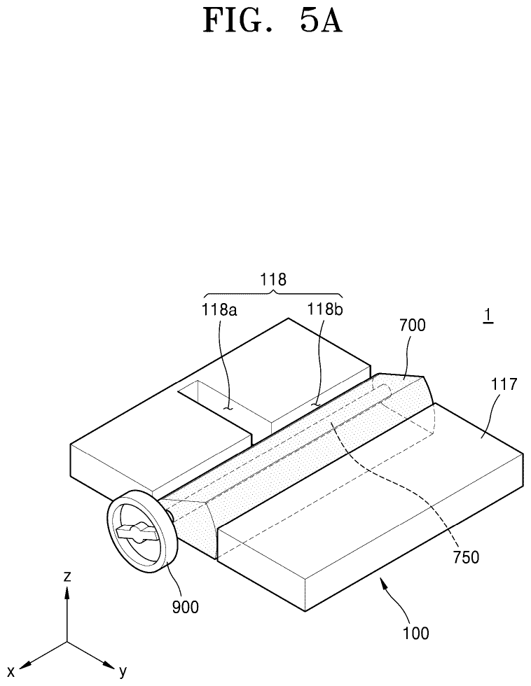

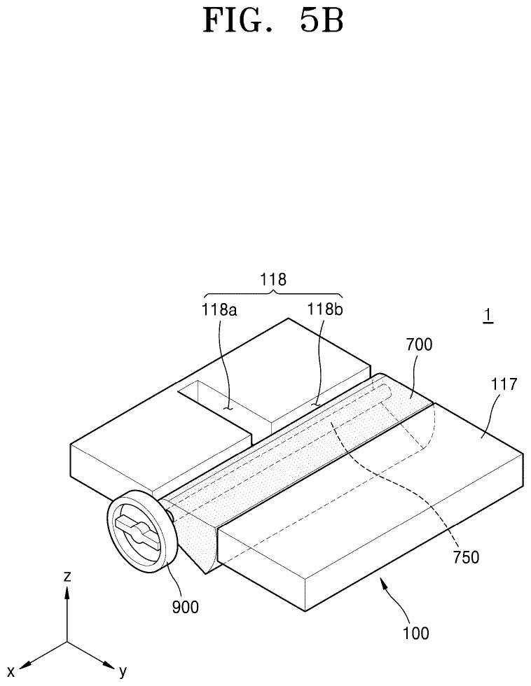

On the other hand, because the locking portion 700 is disposed inside the housing 100, a separate component is required for the user to apply the external force of the locking portion 700. Hereinafter, the lever 900 capable of rotating the locking portion 700 will be described with reference to FIGS. 5A and 5B.

FIGS. 5A and 5B are diagrams for explaining operations of the locking portion 700 and the lever 900 applied to the inhaler 1 according to an embodiment.

Referring to FIGS. 5A and 5B, the inhaler 1 according to an embodiment may include the housing 100, the locking portion 700, and the lever 900.

The lever 900 is a component disposed outside the housing 100 to rotate the locking portion 700. The lever 900 may be connected to the locking portion 700. In this regard, the meaning that the lever 900 is connected to the locking portion 700 may include the lever 900 being directly connected to the locking portion 700, and the lever 900 being connected to the rotating shaft 750 connected to the locking portion 700 and being indirectly connected to the locking portion 700 through the rotating shaft 750.

Referring to FIG. 5A, the locking portion 700 is at a closed position. A user may rotate the locking portion 700 from the closed position to an open position by rotating the lever 900 exposed to the outside of the housing 100.

Referring to FIG. 5B, the locking portion 700 is at the open position. The lever 900 shown in FIG. 5B is in a state in which the lever 900 shown in FIG. 5A has been rotated in a clockwise direction by a certain angle. Accordingly, the locking portion 700 may also rotate by the same angle in the clockwise direction to move from the closed position to the open position.

When the locking portion 700 is at the open position, the locking portion 700 may not protrude to an upper portion of the bottom surface 117. Accordingly, because the locking portion 700 does not interfere with the movement of a push button (e.g., the push button 400 of FIG. 2A), the push button 400 may move along the bottom surface 117.

Meanwhile, the long hole 118 formed in the bottom surface 117 may include a first region 118a extending in a second direction (e.g., y-axis direction) and a second region 118b extending in a third direction (e.g., x-axis direction).

A protrusion (e.g., the protrusion 440 of FIG. 5A) of the push button 400 may be disposed in the first region 118a. The protrusion 440 may move along the first region 118a. The locking portion 700 and the rotation shaft 750 may be disposed in the second region 118b. The second region 118b may have a width (e.g., a length in the y-axis direction) that does not interfere with a rotational motion of the locking portion 700.

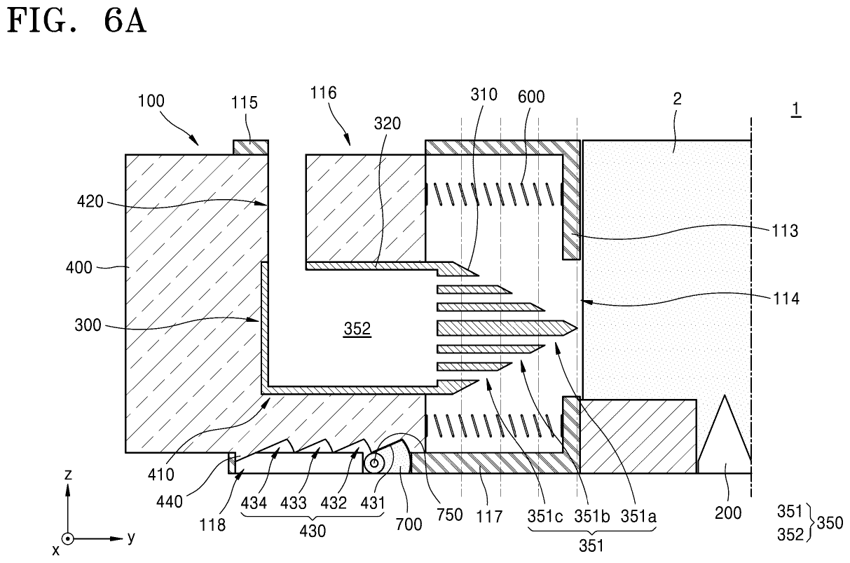

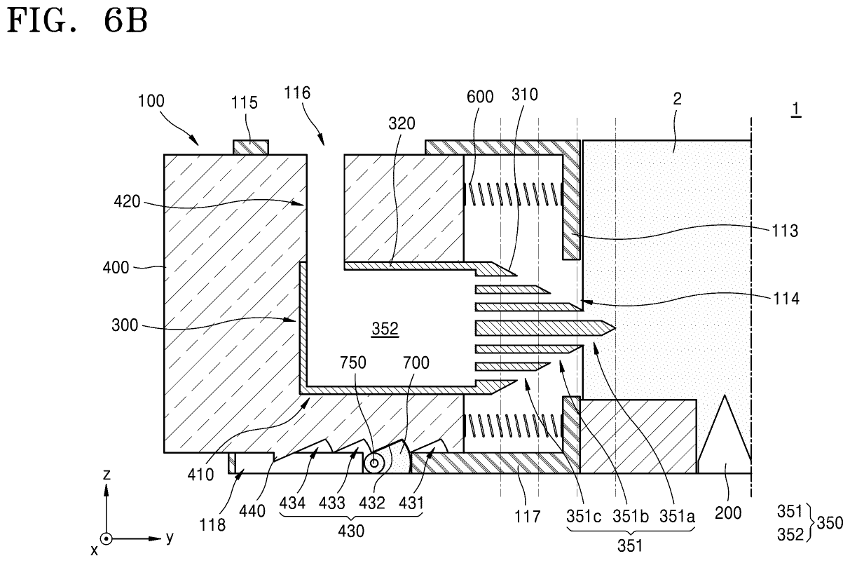

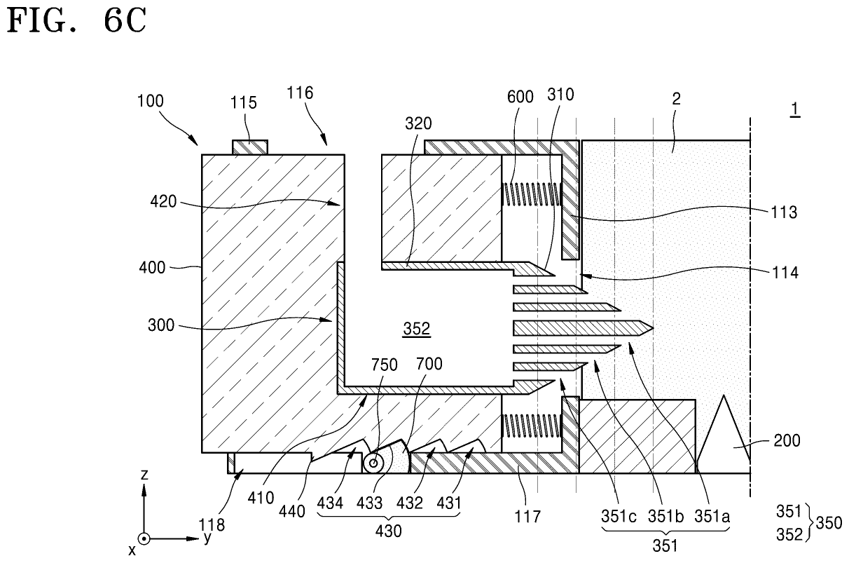

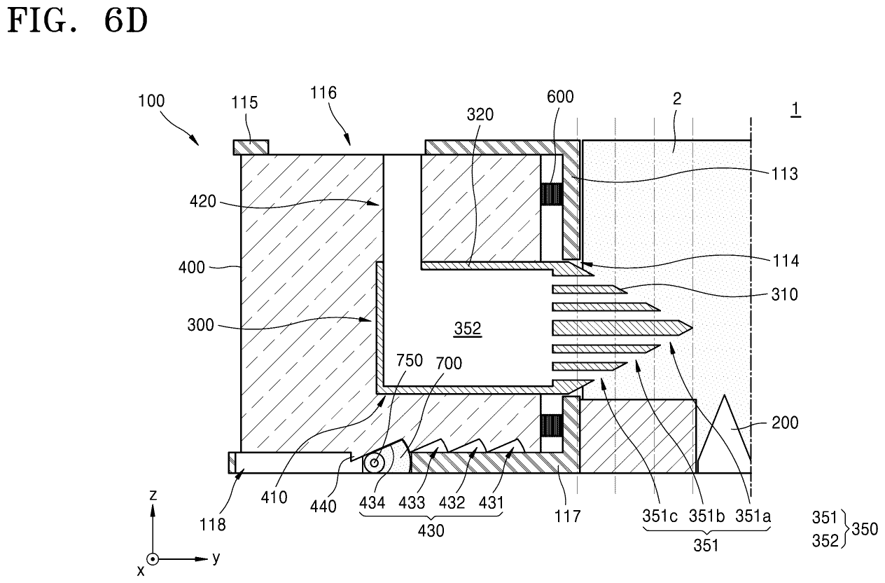

FIGS. 6A to 6D are diagrams for explaining operations of the inhaler 1 according to an embodiment. FIG. 6A shows the second rupture portion 300 at a second position, and FIG. 6B to FIG. 6D show the second rupture portion 300 at a first position.

Referring to FIGS. 6A to 6D, the inhaler 1 according to an embodiment may include the housing 100, the first rupture portion 200, the second rupture portion 300, the push button 400, the first elastic member 600, and the locking portion 700. For convenience of description, some components inside the inhaler 1 are omitted. Detailed descriptions of the configuration and effects of the inhaler 1 which are redundant with those described above will be omitted.

Referring to FIG. 6A, the second rupture portion 300 is spaced apart from the cartridge 2 mounted in the inhaler 1, and the second rupture portion 300 does not rupture the cartridge 2. Because the push button 400 is engaged with the locking portion 700 through a first groove 431, the second rupture portion 300 may be fixed unless a separate external force is applied thereto.

In this regard, the second airflow passage 350 and the inside of the cartridge 2 are not fluidly connected to each other, so even when the user inhales air through a mouthpiece (e.g., the mouthpiece 500 of FIG. 1), no target material may be inhaled.

Referring to FIG. 6B, the rupture part 310 of the second rupture portion 300 is inserted into the cartridge 2, and the second rupture portion 300 ruptures a side surface portion of the cartridge 2. Because the push button 400 is engaged with the locking portion 700 through the second groove 432, the second rupture portion 300 may be fixed unless a separate external force is applied.

In this regard, among the plurality of narrow airflow passages 351, only the first passage 351a may be started in the cartridge 2. In other words, only one end of the first passage 351a among the narrow airflow passages 351 may be disposed inside the cartridge 2. When the user inhales air through the mouthpiece 500, the target material inside the cartridge 2 may move through the first passage 351a.

Referring to FIG. 6C, the rupture part 310 of the second rupture portion 300 is further inserted into the cartridge 2, and the second rupture portion 300 ruptures the side surface portion of the cartridge 2 to be larger than that illustrated in FIG. 6B. Because the push button 400 is engaged with the locking portion 700 through a third groove 433, the second rupture portion 300 may be fixed unless a separate external force is applied thereto.

In this regard, among the plurality of narrow airflow passages 351, the first passage 351a and the second passage 351b may be started inside the cartridge 2. In other words, one end of the first passage 351a and one end of the second passage 351b among the narrow airflow passages 351 may be disposed inside the cartridge 2. When the user inhales air through the mouthpiece 500, the target materials inside the cartridge 2 may move through the first passage 351a and the second passage 351b. Therefore, compared to that illustrated in FIG. 6B, the user may inhale more target materials.

Referring to FIG. 6D, the rupture part 310 of the second rupture portion 300 is further inserted into the cartridge 2, and the second rupture portion 300 ruptures the side surface portion of the cartridge 2 to be larger than that illustrated in FIG. 6C. Because the push button 400 is engaged with the locking portion 700 through a fourth groove 434, the second rupture portion 300 may be fixed unless a separate external force is applied thereto.

In this regard, all the narrow airflow passages 351 may be started inside the cartridge 2. In other words, one end of each of all the narrow airflow passages 351 may be disposed inside the cartridge 2. When the user inhales air through the mouthpiece 500, the target materials inside the cartridge 2 may move through the first passage 351a, the second passage 351b, and the third passage 351c. Therefore, compared to that illustrated in FIG. 6C, the user may inhale more target materials.

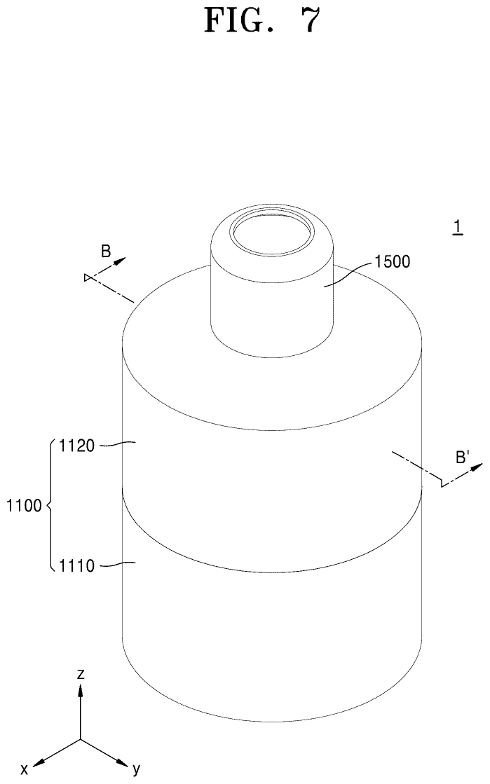

FIG. 7 is a perspective view of the inhaler 1 according to another embodiment.

Referring to FIG. 7, the inhaler 1 according to another embodiment may include a housing 1100 and a mouthpiece 1500. FIG. 7 illustrates the components exposed to the outside of the inhaler 1. Detailed descriptions of the configuration and effects of the inhaler 1 which are redundant with those described above will be omitted.

According to another embodiment, the housing 1100 may have a cylindrical shape. However, the shape of the housing 1100 is not limited to that illustrated. In addition, unlike the embodiment described above, a push button (e.g., the push button 400 of FIG. 1) and a lever (e.g., the lever 900 of FIG. 1) may not be disposed in the inhaler 1 according to another embodiment.

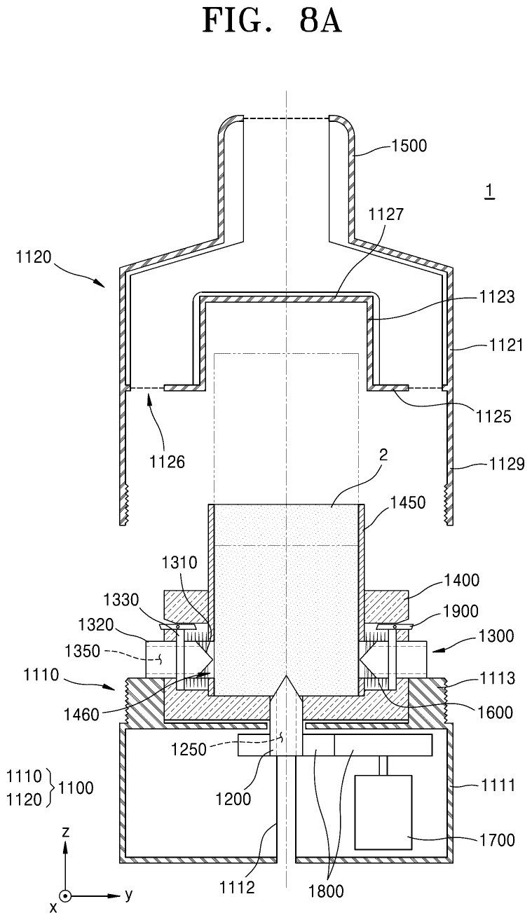

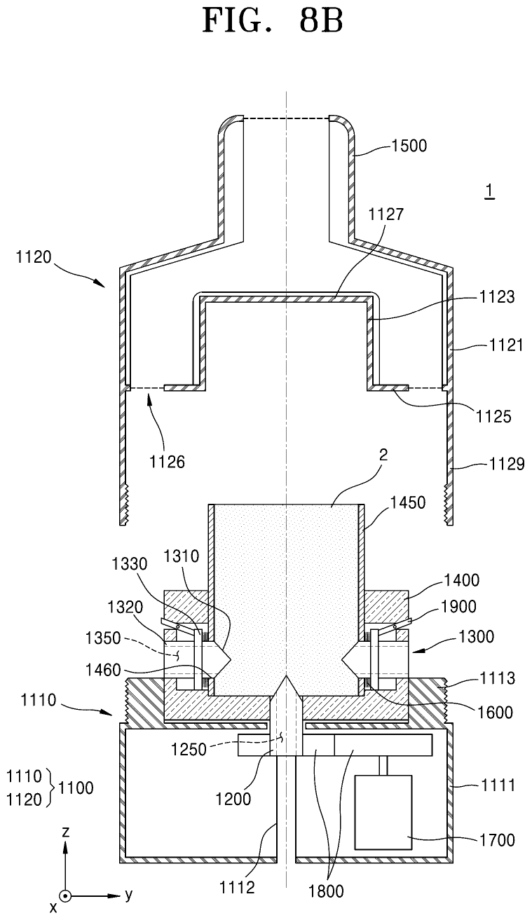

Hereinafter, the internal components of the inhaler 1 according to another embodiment will be described with reference to FIGS. 8A and 8B.

FIGS. 8A and 8B are cross-sectional views of the inhaler 1 according to another embodiment taken along a cross-sectional line B-B′ of FIG. 7, for explaining operations of components disposed inside the inhaler 1.

FIG. 8A shows the second rupture portion 1300 at a second position where the second rupture portion 1300 is spaced apart from a mounting space or the cartridge 2, and FIG. 8B shows the second rupture portion 1300 at a first position where the second rupture portion 1300 protrudes into the mounting space and is inserted into the cartridge 2. In this regard, the mounting space may mean a space surrounded by a support 1450 that is described below.

Referring to FIGS. 8A and 8B, the inhaler 1 according to another embodiment may include the housing 1100, a first rupture portion 1200, a second rupture portion 1300, a rotation unit 1400, the mouthpiece 1500, an elastic member 1600, a driving unit 1700, a power transmission unit 1800, and a rotation button 1900. Detailed descriptions of the configuration and effects of the inhaler 1 which are redundant with those described above will be omitted.

As in the embodiment described above, the housing 1100 may be separated into a lower housing 1110 and an upper housing 1120. The lower housing 1110 may be largely divided into a first part 1111 and a second part 1113 according to a shape. Similarly, the upper housing 1120 may be largely divided into a first part 1121, a second part 1123, a third part 1125, a fourth part 1127, and a fifth part 1129 according to a shape.

The first part 1111 of the lower housing 1110 may be similar to the structure of a first part (e.g., the first part 111 of FIG. 2A) of the embodiment descried above. The first part 1111 may include an inflow passage 1112 extending in a first direction. The inflow passage 1112 may be connected to the first rupture portion 1200.

Meanwhile, the driving unit 1700 and the power transmission unit 1800 may be disposed inside the first part 1111. The driving unit 1700 may rotate a component connected to the driving unit 1700 like a motor. The power transmission unit 1800 may be connected to the driving unit 1700 to rotate. The power transmission unit 1800 rotated by the driving unit 1700 may rotate another power transmission unit 1800 engaged with a gear. Accordingly, a component connected to another power transmission unit 1800 may rotate.

For example, the first rupture portion 1200 may be coupled to another power transmission unit 1800. As the power transmission unit 1800 rotates, the first rupture portion 1200 may also rotate. In this regard, the power transmission unit 1800 and the first rupture portion 1200 may rotate with respect to the same rotation axis. Because the first rupture portion 1200 is coupled to the rotation unit 1400, when the first rupture portion 1200 rotates, the rotation unit 1400 may also rotate.

As another example, the rotation unit 1400 may be coupled to another power transmission unit 1800. As the power transmission unit 1800 rotates, the rotation unit 1400 may also rotate. In this regard, the power transmission unit 1800 and the rotation unit 1400 may rotate with respect to the same rotation axis. Although the first rupture portion 1200 is inserted into the center of the rotation unit 1400, the first rupture portion 1200 may not be coupled to the rotation unit 1400 according to the embodiment. Accordingly, even when the rotation unit 1400 rotates, the first rupture portion 1200 may be fixed. The second part 1113 of the lower housing 1110 is a component extending in a first direction from an upper portion of the first part 1111. The second part 1113 may be disposed to surround the rotation unit 1400. A thread may be disposed on an outer surface of the second part 1113. This may be engaged with the thread disposed in the upper housing 1120 to couple the lower housing 1110 to the upper housing 1120. The upper housing 1120 may be similar to the structure of an upper housing (e.g., the upper housing 120 of FIG. 2A) of the embodiment described above. That is, the descriptions of the first part 1121, the second part 1123, the third part 1125 and the fourth part 1127 of the upper housing 1120 may be the same as those described with reference to FIGS. 2A to 2C.

The fifth part 1129 of the upper housing 1120 has a structure extending downward from the first part 1121. A thread may be disposed on an inner surface of the fifth part 1129. The thread disposed on the outer surface of the second part 1113 of the lower housing 1110 and the thread disposed on the inner surface of the fifth part 1129 of the upper housing 1120 may be engaged with each other so that the lower housing 1110 may be coupled to the upper housing 1120. Accordingly, a separate thread may not be disposed on the second part 1123 of the upper housing 1120.

The first rupture portion 1200 may have the same structure as the first rupture portion 200 of the embodiment described above. The first airflow passage 1250 formed in the first rupture portion 1200 may fluidly connect the inflow passage 1112 to the inside of the cartridge 2. According

to the embodiment, the first rupture portion 1200 may be rotated by the driving unit 1700 and the power transmission unit 1800.

Although the second rupture portion 1300 is functionally similar to the second rupture portion 300 of the embodiment described above, the second airflow passage 1350 may have a structure extending in one direction similar to the first airflow passage 1250.

According to the user's inhalation, air flowing into the cartridge 2 may pass through the second airflow passage 1350 while carrying a target material. The air having passed through the second airflow passage 1350 may flow into a space formed between the upper housing 1120 and the rotation unit 1400. Thereafter, the air may move along the upper housing 1120, pass through an opening 1126 formed in the third part 1125, and then reach the mouthpiece 1500 to be inhaled by the user.

The second rupture portion 1300 may include a rupture part 1310, an extension part 1320, and a protrusion part 1330. The rupture part 1310 and the extension part 1320 may have the same structures as those of the embodiment described above. The protrusion part 1330 is a part with a diameter larger than the diameter of the extending portion 1320 and is in contact with the elastic member 1600.

One end of the elastic member 1600 may be connected to the support 1450, and the other end of the elastic member 1600 may be connected to the protrusion part 1330. The compressed elastic member 1600 may press the protrusion part 1330 in an outward direction of the housing 1100. Accordingly, the second rupture portion 1300 may be pressed in the outward direction of the housing 1100 by the elastic member 1600.

As shown, the elastic member 1600 may be disposed to surround the second rupture portion 1300, but the arrangement of the elastic member 1600 is not limited thereto. As another example, a plurality of elastic members 1600 may be disposed around the second rupture portion 1300 like the first elastic member 600 shown in FIGS. 6A to 6D. Likewise, the first elastic member 600 shown in FIGS. 6A to 6D may be disposed to surround the second rupture portion 300 like the elastic member 1600 shown in FIGS. 8A and 8B.

The rotation unit 1400 is a component for rotating the cartridge 2. At least a part of the mounting space is formed inside the rotation unit 1400, and the cartridge 2 accommodated in the mounting space may rotate together with the rotation unit 1400 according to the rotation of the rotation unit 1400.

In this regard, a central axis of the rotation unit 1400 may be aligned with a central axis of the first rupture portion 1200 in a longitudinal direction. The first rupture portion 1200 may be disposed to penetrate a bottom portion of the rotation unit 1400. Accordingly, the first rupture portion 1200 may rotate together with the rotation unit 1400. The second rupture portion 1300 may also be disposed to penetrate a side surface portion of the rotation unit 1400. Accordingly, the second rupture portion 1300 may also rotate together with the rotation unit 1400.

Meanwhile, the inhaler 1 according to another embodiment may further include the support 1450 for supporting a side surface portion of the cartridge 2. The support 1450 may be disposed inside the rotation unit 1400 to surround a mounting space of the cartridge 2, and support the side surface portion of the cartridge 2 accommodated in the mounting space. In this regard, the space surrounded by the support 1450 may correspond to the mounting space.

As the rotation unit 1400 rotates, the support 1450 may also rotate together with the rotation unit 1400. Because the support 1450 supports the side surface portion of the cartridge 2 and the rotation unit 1400 supports the lower surface of the cartridge 2, when the rotation unit 1400 and the support 1450 rotate, the cartridge 2 may also rotate.

Compared to the rotation unit 1400, the support 1450 may extend longer in the first direction, thereby effectively supporting the side surface portion of the cartridge 2. In addition, a space in which the second rupture portion 1300 and the elastic member 1600 are disposed needs to be formed inside the rotation unit 1400. The support 1450 is disposed in an arrangement space formed by removing a part of the rotation unit 1400 from an inner side of the rotation unit 1400 such that an outer surface of the support 1450 is in contact with an inner surface of the rotation unit 1400, and thus, the convenience of component design may be improved.

However, the embodiment is not necessarily limited to an example where the support 1450 is a separate component from the rotation unit 1400. As another example, the support 1450 may be omitted. As another example, the support 1450 may be treated as the rotation unit 1400 as a sub-component of the rotation unit 1400.

As described above, the elastic member 1600 may be disposed on the outer surface of the support 1450. In addition, the support 1450 may include an opening 1460 opened to allow the second rupture portion 1300 to pass therethrough.

The rotation button 1900 is a component for preventing the second rupture portion 1300 from moving in an outward direction of the housing 1100. When the second rupture portion 1300 moves toward the support 1450 and is inserted into the cartridge 2, the elastic member 1600 connected to the second rupture portion 1300 may press the second rupture portion 1300 in the outward direction of the housing 1100 or the rotation unit 1400. In this regard, the rotation button 1900 may support the second rupture portion 1300 in an inward direction of the housing 1100 to the rotation unit 1400 so that the second rupture portion 1300 remains inserted into the cartridge 2.

The rotation button 1900 may rotate with respect to a rotation axis extending in a third direction (e.g., x-axis direction) crossing the first direction and the second direction. In this regard, a position of the second rupture portion 1300 may be determined according to a position of the rotation button 1900.

Referring to FIG. 8A, the rotation button 1900 is disposed in a longitudinal direction of the second rupture portion 1300 or the second direction. In this regard, the rotation button 1900 may not interfere with the movement of the second rupture portion 1300.

Accordingly, the second rupture portion 1300 may be located in the mounting space or at a second position spaced apart from the cartridge 2 by the pressing of the elastic member 1600. In this regard, because the protrusion part 1330 of the second rupture portion 1300 is in contact with one region of the rotation unit 1400, the movement of the second rupture portion 1300 in the outward direction of the housing 1100 or the rotation unit 1400 may be restricted.

Referring to FIG. 8B, as the second rupture portion 1300 moves toward the support 1450 by a certain distance, there is an extra space for the rotation button 1900 to rotate, and the rotation button 1900 is rotated.

As the rotation button 1900 rotates, the rotation button 1900 is in contact with the protrusion part 1330 of the second rupture portion 1300, and the rotation button 1900 supports the second rupture portion 1300 in the inward direction of the housing 1100 or the rotation unit 1400.

In this regard, despite the pressing of the elastic member 1600, the rotation button 1900 may prevent the second rupture portion 1300 from moving in the outward direction of the housing 1100 or the rotation unit 1400.

Accordingly, the second rupture portion 1300 may be at the first position where pointed one end protrudes into the mounting space. When the cartridge 2 is accommodated in the mounting space, the second rupture portion 1300 may remain inserted into the cartridge 2.

In summary, the user may mount the cartridge 2 on the mounting space surrounded by the support 1450. In this regard, the second rupture portion 1300 may not rupture the cartridge 2 at the second position. Accordingly, the user may not inhale target materials stored in the cartridge 2.

When the user presses the second rupture portion 1300 toward the mounting space or the cartridge 2, the cartridge 2 may be ruptured. The rotation button 1900 which was originally blocked by the protrusion part 1330 and not able to rotate may rotate due to an extra space for rotation according to the movement of the second rupture portion 1300, and as a result, the rotation button 1900 may support the second rupture portion 1300 in the inward direction of the housing 1100 or the rotation unit 1400, and the second rupture portion 1300 may remain inserted into the cartridge 2.

Because the second rupture portion 1300 has ruptured the cartridge 2, the inflow passage 1112, the inside of the cartridge 2, and the mouthpiece 1500 may be fluidly connected to each other by the first airflow passage 250 and the second airflow passage 350, and the user may inhale the target materials stored in the cartridge 2.

When the target materials stored in the cartridge 2 are completely consumed, the user may rotate the rotation button 1900 in a reverse direction to the direction in which the rotation button 1900 has rotated earlier so that the rotation button 1900 no longer supports the protrusion part 1330 of the second rupture portion 1300. Referring to FIG. 8B, the user may rotate the rotation button 1900 in the reverse direction by pressing downward the rotation button 1900 protruding to the outside of the rotation unit 1400.

The second rupture portion 1300 which is no longer supported by the rotation button 1900 may move in the outward direction of the housing 1100 or the rotation unit 1400 by a pressing force of the compressed elastic member 1600. As a result, as shown in FIG. 8A, the second rupture portion 1300 may be separated from the cartridge 2 to be spaced apart from the cartridge 2 or the mounting space. That is, the second rupture portion 1300 may move from the first position to the second position. Because the second rupture portion 1300 is no longer inserted into the cartridge 2, the user may separate the cartridge 2 from the mounting space.

Meanwhile, according to the embodiment, a separate elastic member (not shown) for pressing the rotation button 1900 may be additionally disposed. The elastic member may press the rotation button 1900 to rotate to a position supporting the protrusion part 1330 of the second rupture portion 1300.

In FIG. 8A, although the rotation button 1900 is blocked by the upper portion of the protrusion part 1330 and not able to rotate despite the pressing of the elastic member, in FIG. 8B, as the protrusion part 1330 moves to make a space for the rotation button 1900 to move, the rotation button 1900 may rotate by the elastic member to support the protrusion part 1330

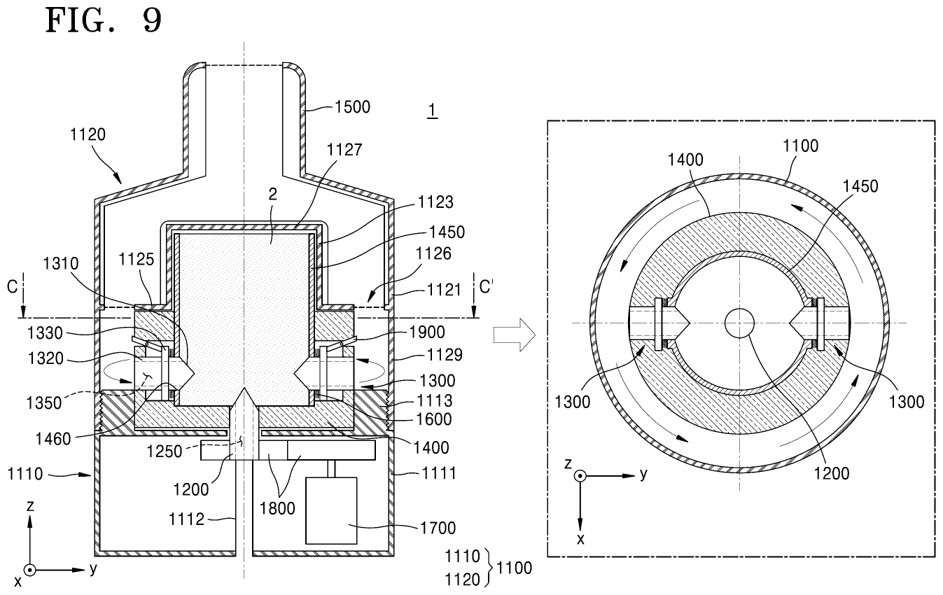

FIG. 9 is a diagram for explaining an internal operating state during use of the inhaler 1 according to another embodiment. In FIG. 9, the left is a cross-sectional view of the inhaler 1 taken along the cross-sectional line B-B′ of FIG. 7, and the right is a cross-sectional view of the inhaler 1 taken along a cross-sectional line C-C′ of the left.

Referring to FIG. 9, the inhaler 1 according to another embodiment may include the housing 1100, the first rupture portion 1200, the second rupture portion 1300, the rotation unit 1400, the mouthpiece 1500, the elastic member 1600, the driving unit 1700, the power transmission unit 1800, and the rotation button 1900. Detailed descriptions of the configuration and effects of the inhaler 1 which are redundant with those described above will be omitted.

FIG. 9 illustrates the second rupture portion 1300 at a first position where the second rupture portion 1300 protrudes into the mounting space and is inserted into the cartridge 2. Accordingly, the inhaler 1 is in a state where a user may inhale target materials stored in the cartridge 2.

In this regard, the rotation unit 1400 may rotate by the driving unit 1700. The first rupture portion 1200, the second rupture portion 1300, the support 1450, the elastic member 1600 and the rotation button 1900 may rotate with respect to the same rotation axis together with the rotation unit 1400.

As the rotation unit 1400 rotates, the cartridge 2 may also rotate. The target materials stored inside the cartridge 2 may move toward the edge of the cartridge 2 by a centrifugal force by rotation. In this regard, because the second rupture portion 1300 ruptures a side surface portion of the cartridge 2, the centrifugal force by rotation may promote the target materials to be discharged to the outside of the cartridge 2 through the second airflow passage 1350.

Accordingly, when the user inhales the target materials through the mouthpiece 1500, even when an inhalation pressure is somewhat insufficient, because the target materials are discharged to the outside of the cartridge 2, the user may easily inhale the target materials even with a low inhalation pressure.

Meanwhile, the rotation of the rotation unit 1400 by the driving unit 1700 may be controlled by a control unit (not shown). When the user issues a command to the control unit so that the rotation unit 1400 rotates through an input unit (not shown), the control unit may control the driving unit 1700 to rotate the rotation unit 1400.

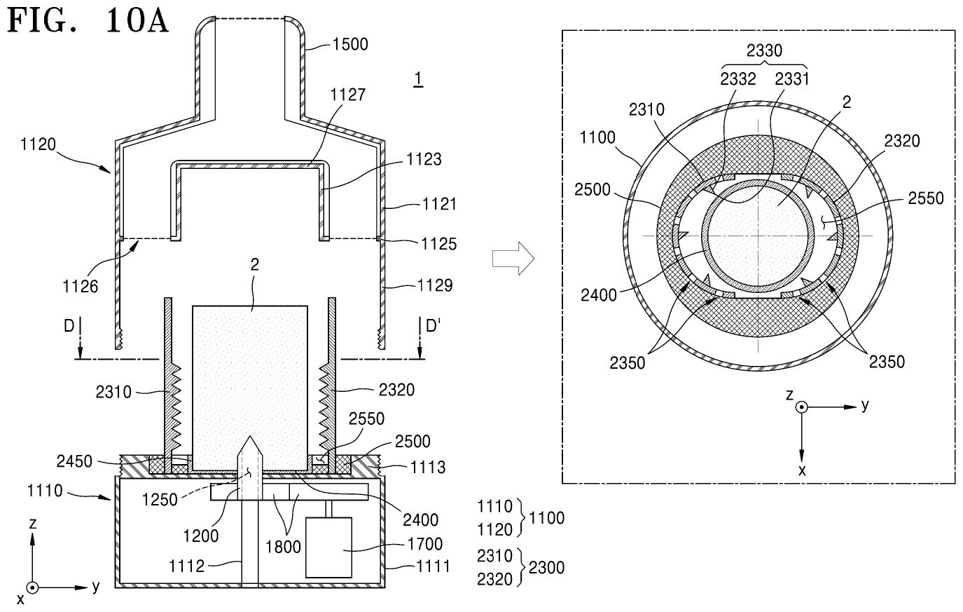

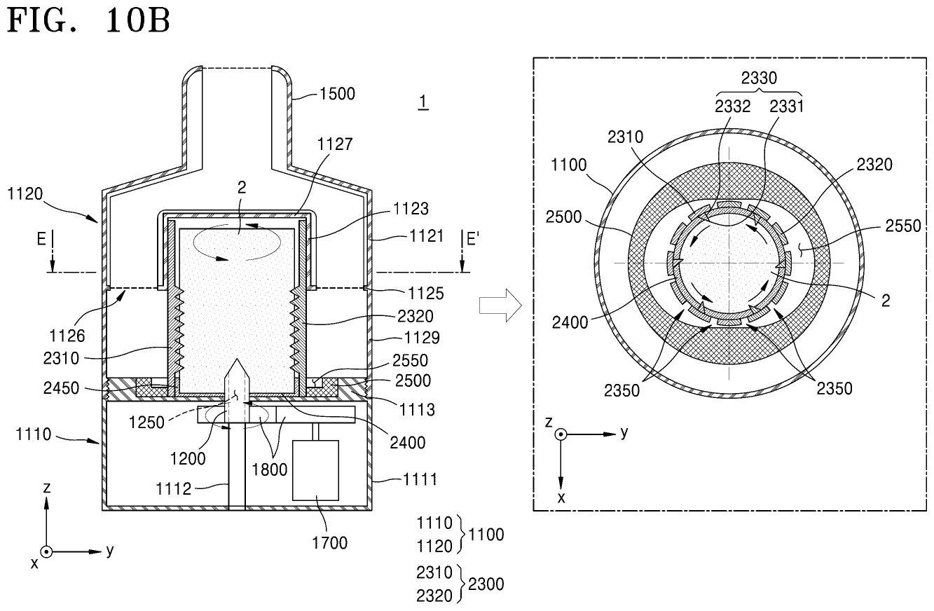

FIG. 10A and FIG. 10B are diagrams for explaining a rupture process of a cartridge mounted in the inhaler 1 according to another embodiment and an internal operation state during use of the inhaler 1. In FIGS. 10A and 10B, the left is a cross-sectional view of the inhaler 1 taken along the cross-sectional line B-B′ of FIG. 7, and the right is a cross-sectional view of the inhaler 1 taken along cross-sectional lines D-D′ and E-E′ of the left.

FIG. 10A illustrates a second rupture portion 2300 at a second position where the second rupture portion 2300 is spaced apart from a mounting space or the cartridge 2, and FIG. 10B illustrates the second rupture portion 2300 at a first position where the second rupture portion 2300 protrudes into the mounting space and is inserted into the cartridge 2. In this regard, the mounting space may mean a space surrounded by a support 2450 that is described below.

Referring to FIGS. 10A and 10B, the inhaler 1 according to another embodiment may include the housing 1100, the first rupture portion 1200, the mouthpiece 1500, the driving unit 1700, the power transmission unit 1800, the second rupture portion 2300, a rotation unit 2400, and a guide portion 2500. Detailed descriptions of the configuration and effects of the inhaler 1 which are redundant with those described above will be omitted.

According to another embodiment, the housing 1100, the first rupture portion 1200, the mouthpiece 1500, the driving unit 1700, and the power transmission unit 1800 are the same as described above, and thus descriptions thereof will be omitted.

However, unlike the previous embodiment, the second rupture portion 2300 may be disposed in the form of rupture structures 2310 and 2320 in the inhaler 1 according to another embodiment. That is, the second rupture portion 2300 may include the first rupture structure 2310 and the second rupture structure 2320.

The second rupture portion 2300 may be separated into the first rupture structure 2310 and the second rupture structure 2320 having shapes symmetrical to each other. In this regard, the first rupture structure 2310 and the second rupture structure 2320 may be separated in a direction away from each other in a second direction crossing a longitudinal direction of the housing 1100.

One or more rupture protrusions 2330 including a fine point element 2331 and a blade element 2332 may be disposed on each of the first rupture structure 2310 and the second rupture structure 2320. The rupture protrusion 2330 may be disposed on an inner surface of each of the rupture structures 2310 and 2320.

The fine point element 2331 of the rupture protrusion 2330 may protrude into the mounting space to perforate a side surface portion of the cartridge 2. The blade element 2332 of the rupture protrusion 2330 may be disposed to face a circumferential direction of the cartridge 2, and cut the cartridge 2 while the cartridge 2 rotates by the rotation unit 2400.

As shown, a plurality of rupture protrusions 2330 may be disposed in a longitudinal direction of the rupture structures 2310 and 2320 or a first direction. In addition, the plurality of rupture protrusions 2330 may be disposed in a circumferential direction of the rupture structures 2310 and 2320. However, the number and arrangement of the rupture protrusions 2330 are not limited to those illustrated. As another example, the rupture protrusion 2330 may be disposed in an elongated shape such as a thread.

One or more second airflow passages 2350 may be disposed in the rupture structures 2310 and 2320. As shown, a plurality of second airflow passages 2350 may be spaced apart at equal intervals in the circumferential direction of the rupture structures 2310 and 2320, together with the rupture protrusion 2330, but the number and arrangement of second airflow passages 2350 are not limited to those illustrated.

The first rupture structure 2310 and the second rupture structure 2320 may be coupled to each other to function as a single second rupture portion 2300. The second rupture portion 2300 in which the first rupture structure 2310 and the second rupture structure 2320 are coupled to each other may have a hollow column shape (e.g., a cylindrical shape) extending in the first direction. The second rupture portion 2300 may surround the mounting space into which the cartridge 2 is inserted. The rupture protrusion 2330 formed inside the second rupture portion 2300 may be inserted into the cartridge 2 to rupture the cartridge 2.

In this regard, when the second rupture portion 2300 is always in the form of the first rupture structure 2310 and the second rupture structure 2320 which are coupled to each other, there may be a problem that the cartridge 2 is blocked by the rupture protrusion 2330 and not inserted into a bottom surface of the mounting space when inserted into the mounting space.

In order to prevent such a problem, the second rupture portion 2300 may be separated into the first rupture structure 2310 and the second rupture structure 2320. Referring to FIG. 10A, the first rupture structure 2310 and the second rupture structure 2320 may move in an outward direction of the housing 1100 or the rotation unit 2400 and be separated. The separated first rupture structure 2310 and second rupture structure 2320 may include a profile or channel bar having a cross-sectional shape of a ‘C’ or ‘U’.

In this regard, the rupture protrusion 2330 may be at a second position spaced apart from the mounting space or the cartridge 2. Referring to FIG. 10B, the first rupture structure 2310 and the second rupture structure 2320 may move in an inward direction of the housing 1100 or the rotation unit 2400 and be coupled. In this regard, the rupture protrusion 2330 may be at a first position where the rupture protrusion 2330 protrudes into the mounting space and is inserted into the cartridge 2.

Therefore, when the second rupture portion 2300 is away from the mounting space and separated into the first rupture structure 2310 and the second rupture structure 2320, the cartridge 2 may be inserted into the mounting space. When the first rupture structure 2310 and the second rupture structure 2320 move toward the mounting space and are coupled to each other, the second rupture portion 2300 may rupture the cartridge 2 accommodated in the mounting space. In order for the first rupture structure 2310 and the second rupture structure 2320 to be separated from or coupled to each other, the first rupture structure 2310 and the second rupture structure 2320 need to move, respectively.

In this regard, the inhaler 1 according to another embodiment may further include the guide portion 2500 such that the first rupture structure 2310 and the second rupture structure 2320 may move only in one direction. The guide portion 2500 may be disposed outside the rotation unit 2400 and guide movement between the first position and the second position of the second rupture portion 2300.

That is, the guide portion 2500 may guide the movement of the first rupture structure 2310 and the second rupture structure 2320 even when the first rupture structure 2310 and the second rupture structure 2320 are away from the mounting space and separated, and even when the first rupture structure 2310 and the second rupture structure 2320 move toward the mounting space and are coupled.

In this regard, the guide portion 2500 may include a guide groove 2550. a lower portion of each of the first rupture structure 2310 and the second rupture structure 2320 may be inserted into the guide groove 2550 so as not to deviate from the guide portion 2500.

According to another embodiment, the rotation unit 2400 may have a relatively simple structure compared to the rotation unit 1400. As the rotation unit 2400 rotates by the driving unit 1700, the cartridge 2 located on the rotation unit 2400 may rotate together with the rotation unit 2400. In this regard, the first rupture portion 1200 may rotate together with the rotation unit 2400 or be fixed according to an embodiment.

According to another embodiment, the support 2450 may exist as a sub-component of the rotation unit 2400. That is, the support 2450 may correspond to a part of the rotation unit 2400. The support 2450 may include a hollow column shape (e.g., a cylindrical shape) extending in the first direction from the edge of the rotation unit 2400.

The support 2450 may surround the mounting space of the cartridge 2 and support the side surface portion of the cartridge 2 accommodated in the mounting space. In this regard, a region surrounded by the support 2450 may correspond to the mounting space.

According to another embodiment, when the rotation unit 2400 rotates by the driving unit 1700, the first rupture portion 1200 and the support 2450 may rotate together with the rotation unit 2400 with respect to the same rotation axis. In this regard, because the support 2450 is a part of the rotation unit 2400, only the first rupture portion 1200 and the rotation unit 2400 may rotate. According to an embodiment, when the first rupture portion 1200 also does not rotate, only the rotation unit 2400 among the components inside the inhaler 1 may rotate.

As the rotation unit 2400 rotates, the cartridge 2 rotates together with the rotation unit 2400, but the rupture protrusion 2330 inserted into the cartridge 2 and the second rupture portion 2300 including the rupture protrusion 2330 may remain stopped. Accordingly, the second airflow passage 2350 formed on the second rupture portion 2300 may also be at a fixed position. In addition, the guide portion 2500 disposed outside the rotation unit 2400 may also remain stopped while the rotation unit 2400 rotates.

Accordingly, the rotating cartridge 2 may be cut by the blade element 2332 formed on the rupture protrusion 2330. That is, as the cartridge 2 rotates, a ruptured part may be larger. The size of the ruptured part of the cartridge 2 may vary depending on the shape of the rupture protrusion 2330.

As described above, the centrifugal force by rotation may promote target materials to be discharged to the outside of the cartridge 2 through a ruptured region of the side surface portion of the cartridge 2. Because the plurality of second airflow passages 2350 are formed at various positions on the second rupture portion 2300, the target materials discharged to the outside of the cartridge 2 may pass through the second airflow passages 2350 and discharged to one region surrounded by the housing 1100.

Accordingly, when the user inhales the target materials through the mouthpiece 1500, even when an inhalation pressure is somewhat insufficient, because the target materials are discharged to the outside of the cartridge 2, the user may easily inhale the target materials even with a low inhalation pressure.

According to the inhaler according to the embodiments, the sealed cartridge may be conveniently and stably ruptured by using the internal structure of the inhaler.

In addition, according to the inhaler according to the embodiments, unnecessary discharging of the target materials may be minimized.

In addition, according to the inhaler according to the embodiments, the target materials may be easily inhaled even with a low inhalation pressure.

Certain embodiments or other embodiments of the present disclosure described above are not exclusive or distinct from each other. The certain embodiments or other embodiments of the present disclosure described above may be combined with each other or used in combination with each other in their respective components or functions.

For example, it means that an A component described in a specific embodiment and/or the drawings and a B component described in another embodiment and/or the drawings may be combined with each other. In other words, even when it is not explained directly about combination between components, it is possible to combine unless it is explained that combination is impossible.

The above detailed description should not be interpreted restrictedly but should be considered illustrative in all aspects. The scope of the present disclosure should be determined by a rational interpretation of the attached claims, and all changes within the equivalent scope of the present disclosure are included in the scope of the present disclosure.

According to the inhaler according to the embodiments, the sealed cartridge may be conveniently and stably ruptured by using the internal structure of the inhaler.

In addition, according to the inhaler according to the embodiments, unnecessary discharging of the target materials may be minimized.

Effects of the present disclosure are not limited to the above effects, and effects that are not mentioned could be clearly understood by one of ordinary skill in the art from the present specification and the attached drawings.

What is claimed is:

1. An inhaler comprising:
a housing comprising a mounting space to accommodate a cartridge storing target materials; and
a rupture portion comprising an airflow passage and configured to rupture the cartridge,
wherein the rupture portion comprises
a first rupture portion extending in a first direction and having pointed one end protruding in the mounting space, wherein the first direction is a longitudinal direction of the housing; and
one or more second rupture portions extending in a second direction crossing the first direction and being movable in the second direction between a first position where the pointed one end protrudes into the mounting space and a second position spaced apart from the mounting space.

2. The inhaler of claim 1, wherein
the housing includes a lower housing and an upper housing, the lower housing surrounding at least a part of an outer surface of the cartridge and including the mounting space that is opened in the first direction and the upper housing surrounding an upper portion of the cartridge, and
the lower housing and the upper housing are separably coupled to each other.

3. The inhaler of claim 1, further comprising: a push button protruding to outside of the housing through a side surface portion of the housing,
wherein the one or more second rupture portions are coupled to the push button and moves together with the push button.

4. The inhaler of claim 3, wherein
the push button includes a first opening part that is opened in the second direction and a second opening part that is opened in the first direction,
the one or more second rupture portions are inserted into the push button through the first opening part, and
airflow passage of each of the one or more second rupture portions is fluidly connected to the second opening part.

5. The inhaler of claim 3, further comprising: a first elastic member pushing the push button in an outward direction of the housing,
wherein one end of the first elastic member is connected to one region of the housing surrounding the mounting space and another end of the first elastic member is connected to the push button.

6. The inhaler of claim 3, further comprising:
a locking portion protruding on a movement path of the push button to fix the push button; and
a second elastic member pressing the locking portion to remain protruding on the movement path of the push button,
wherein the push button includes one or more grooves engaged with the locking portion.

7. The inhaler of claim 6, wherein
the locking portion is movable between a closed position protruding on the movement path of the push button and fixing the push button and an open position spaced apart from the movement path of the push button and allowing the movement of the push button,
the inhaler further comprising: a lever connected to the locking portion and exposed to the outside of the housing to allow a user to move the locking portion to the open position.

8. The inhaler of claim 1, wherein
each of the one or more second rupture portions includes a rupture part that narrows toward the one end of each of the one or more second rupture portions and an extension part extending with a same diameter as the other end of each of the one or more second rupture portions, and
the airflow passage of each of the one or more second rupture portions includes one or more narrow airflow passages extending from one end of the rupture part in the second direction and a wide airflow passage extending toward one end of the extension part and combined with the one or more narrow airflow passages.

9. The inhaler of claim 8, wherein
when the one or more second rupture portions are at the first position,
at least a part of the rupture part is inserted into the cartridge, and
at least one of the one or more narrow airflow passages starts inside the cartridge.

10. The inhaler of claim 8, wherein
the one or more narrow airflow passages are disposed in plural, and
the plurality of narrow airflow passages include first passages starting at a position spaced apart from the pointed one end of the rupture part by a first distance and second passages starting at a position spaced apart from the pointed one end by a second distance greater than the first distance, and
a number of the second passages is greater than or equal to a number of the first passages.

11. The inhaler of claim 1, further comprising: a rotation unit rotating the cartridge accommodated in the mounting space.

12. The inhaler of claim 11, wherein
at least a part of the mounting space is formed inside the rotation unit, and
the one or more second rupture portions are disposed to penetrate a side surface portion of the rotation unit and rotates together with the rotation unit.

13. The inhaler of claim 11, further comprising:
a support disposed inside the rotation unit to surround the mounting space and support a side surface portion of the cartridge accommodated in the mounting space;
an elastic member having one end connected to the support and another end connected to any one of the one or more second rupture portions to press the one of the one or more second rupture portions in an outward direction of the rotation unit; and
a rotation button supporting the at least one second rupture portion in an inward direction of the rotation unit so that the at least one second rupture portion moves toward the support and remains inserted into the cartridge.

14. The inhaler of claim 11, wherein
each of one or more second rupture portions has a hollow column shape extending in the first direction,
each of one or more second rupture portions is separable into a first rupture structure and a second rupture structure having shapes symmetrical to each other, and
the first rupture structure and the second rupture structure are separated in a direction away from each other in the second direction.

15. The inhaler of claim 11, wherein
the at least one second rupture portion includes a blade element facing a circumferential direction of the cartridge, and
the at least one second rupture portion is fixed at the first position while the rotation unit rotates.
